(19) United States
(12) Patent Application Publication
(10) Pub. No.: US 2010/0055009 A1
(43) Pub. Date: Mar. 4, 2010

(54) TREATING CHAMBER FOR HIGH-STRENGTH PULSE ELECTRIC FIELD AND HIGH-FREQUENCY ELECTRIC FIELD TREATING EQUIPMENT

(76) Inventor: Jinquan Chen, Fujian Province (CN)

Correspondence Address: DAVID AND RAYMOND PATENT FIRM, 108 N. YNEZ AVE., SUITE 128, MONTEREY PARK, CA, 91754, US

(21) Appl. No.: 12/312,657

(22) PCT Filed: Dec. 4, 2007
(86) PCT No.: PCT/CN2007/003436

§ 371 Date: May 19, 2009

(30) Foreign Application Priority Data
Jan. 12, 2007 (CN) 200710008437.X

(51) Int. Cl. A61K 39/02

(52) U.S. Cl. 422/243

(57) ABSTRACT

It's referred to a continuous fluid treatment chamber designed for pulsed electric field or radio frequency electric field. It's a chamber with two metal hollow tubes and isolating ring. Isolating ring is between two metal hollow tubes which are connected with high intensive pulse or radio frequency generator respectively. The main characteristics are as follow: In two metal hollow tubes near the isolating ring there is at least one metal conductor or lead. With this chamber, the capacity can be greatly improved (the material can pass through any large diameter tube, not sew). And the electric field distribution can be more uniform and the treatment of fluid will be highly efficient. It's benefit for pulse electric field or radio frequency electric field to be applied in industry.

### BACKGROUND OF THE PRESENT INVENTION

1. Field of Invention

This patent is referred to a continuous fluid treatment chamber designed for pulsed electric field or radio frequency electric field which is mainly applied in liquid food such as fruit and vegetable juice sterilization and enzyme inhibition, pretreatment of bottled drinking water, aging process of brewing, separation and extraction of effective components or other fields relative to liquid treatment like wastewater treatment or water purification. The invented chamber can be used in different electric field including pulse electric field, radio frequency electric field and so on.

2. Description of Related Arts

At present, the treatment chamber of pulsed electric field or radio frequency electric field can only be used in lab and is far from industry. The main problems are as follow: the section acreage was too small for flow and the electric field distribution is not uniform which will cause two bad effects: some parts are easily to discharge and some parts are too low to have effect on treatments.

The common treatment chamber (FIG. 2 without metal conductor or lead) is made of two metal hollow tubes (1) which are connected with high intensive pulse or radio frequency generator respectively and isolating ring (2) between them. Its electric field distribution is showed in FIG. 4. The electric field along the tube wall is densest which means highest strength. With distance to the wall, the equipotential line in the middle of tube is spare. It's more obvious when the diameter of tube enlarges. As the flow rate in the middle is fastest cutting down the effect of electric field, which will restrict the application of pulse electric field or radio frequency electric field.

In order to change the above problem of chamber, it was revealed a sort of processing equipment of high-voltage pulsed electric fields by Chinese patent of invention whose number was 200410011305.9. Its improvements included: an insulating bar whose diameter was less than the insulated isolating collar was inserted in those two mental hollow tubes. Through the improvement, the fluid can flow along the approaching pipe wall to the processing place at the polar juncture, and it can avoid problems of the sparseness of central isothermal line of pipe and the poor processing effect due to the maximum flow rate of fluid, to make the fluid flow only along the closing to pipe wall which had the maximum electrical field, reaching a better processing effect. But it minimized the sectional area of fluid flowing, and the fluid flowing was only within the scope of millimeter.

### SUMMARY OF THE PRESENT INVENTION

In order to solve the problem mentioned above, a continuous fluid treatment chamber designed for pulsed electric field or radio frequency electric field is provided. With this chamber, the capacity can be greatly improved (the material can pass through any large diameter tube, not sew). And the electric field distribution can be more uniform and the treatment of fluid will be highly efficient. It's benefit for pulse electric field or radio frequency electric field to be applied in industry.

It's referred to a chamber with two metal hollow tubes and isolating ring. Isolating ring is between two metal hollow tubes which are connected with high intensive pulse or radio frequency generator respectively. The main characteristics are as follow: In two metal hollow tubes near the isolating ring there is at least one metal conductor or lead.

Compared with other technology, in this patent through adding metal conductor or lead in tubes, the equipotential line in tube will be more uniform and denser which will not restrict the diameter of tubes. It will not only improve the turnover, but also benefit for pulse electric field or radio frequency electric field to be applied in industry.

In above method, the ends of metal conductors are welded with metal tubes. The number of conductor or lead can be increased with larger diameter of tube.

In order to make the equipotential line more uniform and denser, the conductor or lead would better be vertical to axes of metal tubes. Of course, among the small angle scope, it'll not affect the proportion and density of distribution.

When the material is food, in order to avoid rust, the tubes, conductor or lead would better be stainless steel or noble metal(such as gold foil) which is safe for food.

The metal conductor or lead in two metal hollow tubes can be mutual parallel or set by any angle.

These and other objectives, features, and advantages of the present invention will become apparent from the following detailed description, the accompanying drawings, and the appended claims.

### BRIEF DESCRIPTION OF THE DRAWINGS

FIG. 1 is the treatment of material (can be changed based on application fields of pulsed electric field or radio frequency electric field).

FIG. 2 is a Sketch map of treatment chamber referred by patent.

FIG. 3 is a side elevation of FIG. 2.

FIG. 4 is the line in treatment chamber without metal conductor or lead.

FIG. 5 is the equipotential line in treatment chamber with one pair of metal conductors or leads.

FIG. 6 is the equipotential line in treatment chamber with three pair of metal conductors or leads.

FIG. 7 is the equipotential line in treatment chamber with five pair of metal conductors or leads.

FIG. 8 is the equipotential line in treatment chamber with three metal conductors or leads in positive pole across with one metal conductor or lead in negative pole.

FIG. 9 is an electric field distribution of treatment chamber without metal conductor or lead.

FIG. 10 is an electric field distribution of treatment chamber with five pair of metal conductors or leads referred in this patent.

FIG. 11 is the relative survival rates of microorganism in different parts along diameter direction of treatment chamber before and after improvement.

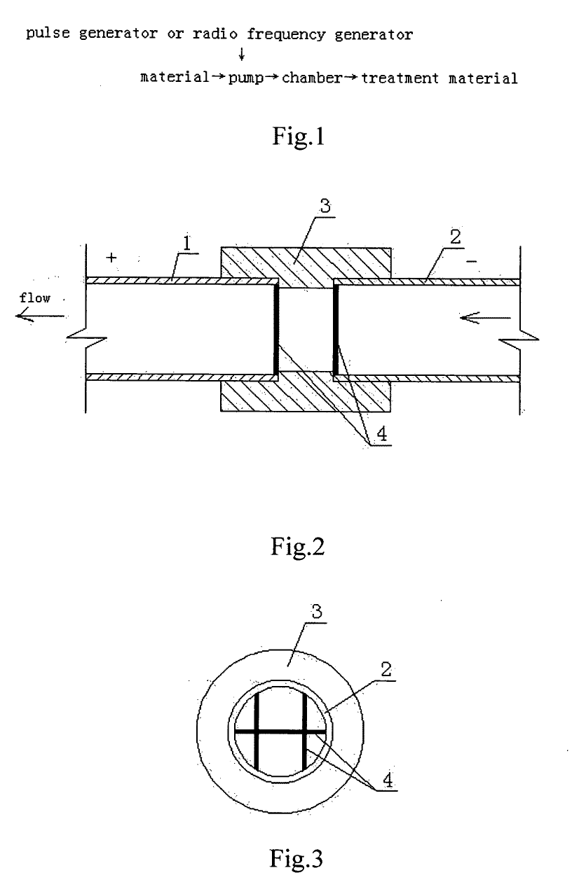

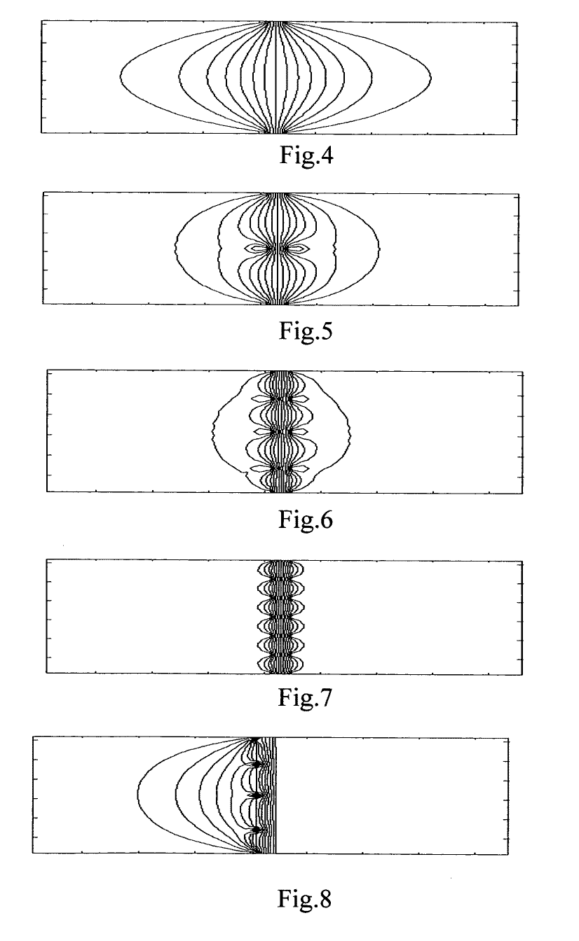

### DETAILED DESCRIPTION OF THE PREFERRED EMBODIMENT

The specification combining figures and implementing can be described as follow: As FIGS. 2 and 3 show, a continuous treatment chamber consists of two metal hollow tubes 1, 2 and isolating ring 3 (made of polytetrafluoroethylene). Isolating ring is between two metal hollow tubes which are connected with high intensive pulse or radio frequency generator respectively. The main characteristics are as follow: In two metal hollow tubes near the isolating ring there is at least one metal conductor or lead 4 (When the material is food, in order to avoid rust, the tubes, conductor or lead would better be stainless steel or noble metal (such as gold foil) which is safe for food; When it's used to treat other liquid, the material can be other metal.). The ends of metal conductors are welded with metal tubes. It can be connected electrically by other methods. The conductor or lead would better be vertical to axes of metal tubes. The metal conductor or lead in two metal hollow tubes can be mutual parallel or set by any angle. The shape of metal conductor or lead can be cylinder, lozenge, cuboid or various shapes. The cross section of conductor or lead can be either big or small, and the shape and thickness can be adjusted according to necessary. The number of conductor or lead can be 1, 2 or more. The number of conductor or lead can be increased with larger diameter of tube. The interspace between two metal tubes and isolating ring is the chamber for pulsed electric field or radio frequency electric field. The material passes through from one tube to the other.

To get the sterilization effect, the strength of pulsed electric field should be more than 5 kV/cm and the frequency 10 Hz to 3000 Hz. The strength of radio frequency electric field should be more than 0.2 kV/cm and the frequency 20 kHz to 50 MHz. When the parameters are below the value mentioned above, the effect is bad. While the frequency is above, it will provide high request for the device.

FIGS. 5, 6, 7 show the equipotential line distribution in treatment chamber with one, three, five pair of metal conductors or leads referred in this patent. The adding metal conductor or lead changes the equipotential line distribution, enhances the strength in the middle of chamber and improves the treatment efficiency.

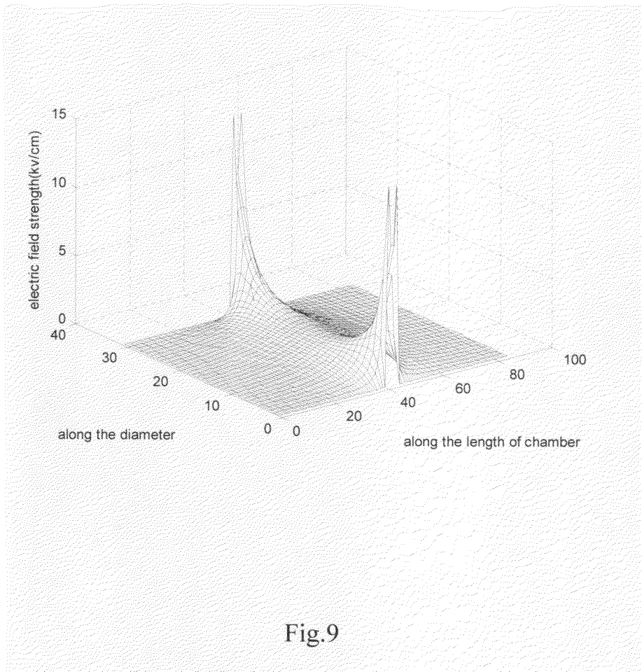

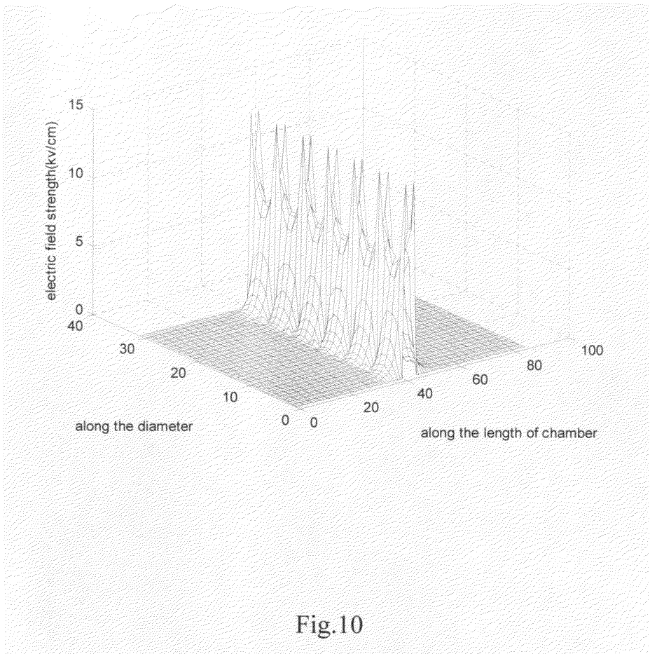

FIG. 8 shows the equipotential line in treatment chamber with three metal conductors or leads in positive pole across with one metal conductor or lead in negative pole. The angle between two tubes is adjusted to make the conductors cross. Thus, one side of equipotential line is compressed completely and a uniform distribution is set up in the middle of tube.

FIG. 9 shows electric field distribution of unimproved treatment chamber. The strength on the wall is highest, while in the middle is weakest.

FIG. 10 shows electric field distribution of treatment chamber with five pair of metal conductors or leads. The strength on the wall and in the middle is both high and uniform.

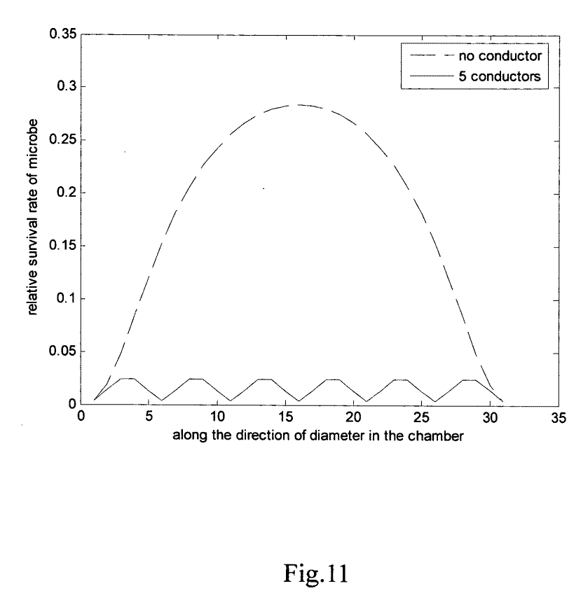

In FIG. 11 dashed means the relative survival rate of microorganism in unimproved chamber, which is low near the wall and high in the middle. Real line means the relative survival rate of microorganism in improved chamber, which is low both near the wall and in the middle.

All in all, by adding the conductors in the tubes, the distribution in the chamber is improved which will no longer restrict the diameter of chamber. No matter how large the tubes are, the ideal strength can be obtained by combination and arrangement of conductors. The capacity can be greatly improved by this patent. It is mainly applied in liquid food such as fruit and vegetable juice sterilization and enzyme inhibition, pretreatment of bottled drinking water, aging process of brewing, separation and extraction of effective components or other fields relative to liquid treatment like wastewater treatment or water purification. It's benefit for pulse electric field or radio frequency electric field to be applied in industry.

One skilled in the art will understand that the embodiment of the present invention as shown in the drawings and described above is exemplary only and not intended to be limiting.

It will thus be seen that the objects of the present invention have been fully and effectively accomplished. The embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A treating chamber for high-strength pulse electric field and high-frequency electric field fluid treating equipment, wherein said includes: consists of two metal hollow tubes and isolating ring, isolating ring is between two metal hollow tubes which are connected with high intensive pulse or radio frequency generator respectively, wherein in two metal hollow tubes near the isolating ring there is at least one metal conductor or lead.

2. The treating chamber for high-strength pulse electric field and high-frequency electric field fluid treating equipment according to claim 1, wherein the ends of metal conductors are welded with metal tubes.

3. The treating chamber for high-strength pulse electric field and high-frequency electric field fluid treating equipment according to claim 1 or 2, wherein the mentioned metal conductor or lead is vertical to axes of metal tubes.

4. The treating chamber for high-strength pulse electric field and high-frequency electric field fluid treating equipment according to claim 3, wherein the mentioned metal conductor and lead are made of stainless steel.

5. The treating chamber for high-strength pulse electric field and high-frequency electric field fluid treating equipment to claim 3, wherein the metal conductor or lead in two metal hollow tubes is mutual parallel.

6. The treating chamber for high-strength pulse electric field and high-frequency electric field fluid treating equipment to claim 3, wherein the metal conductor or lead in two metal hollow tubes is set by any angle.
